(19) United States
(12) Patent Application Publication
(10) Pub. No.: US 2016/0193607 A1
(43) Pub. Date: Jul. 7, 2016

(54) TRANSPORTABLE COMPOSITE LIQUID CELLS

(71) Applicant: GENCELL BIOSYSTEMS LTD., Raheen, County Limerick (IE)

(72) Inventors: Brian Barrett, Cashel, County Tipperary (IE); David McGuire, Enniscorthy, County Wexford (IE); Robert Roeven, Stoughton, WI (US)

(21) Appl. No.: 14/916,947

(22) PCT Filed: Nov. 25, 2014
(86) PCT No.: PCT/IB2014/003058

§ 371 Date: Mar. 4, 2016

Related U.S. Application Data
(60) Provisional application No. 61/908,479, filed on Nov. 25, 2013.
(60) Provisional application No. 61/908,489, filed on Nov. 25, 2013.

(51) Int. Cl. B01L 3/00; C12Q 1/68; G01N 35/10; B01L 7/00

(52) U.S. Cl. CPC B01L 3/527; B01L 7/52; C12Q 1/686; C12Q 1/6806; G01N 35/1009; B01L 2300/044; B01L 2200/14; B01L 2300/0672; B01L 2300/0627; B01L 2300/18; B01L 2300/1894; B01L 2300/0838; B01L 2200/16

(57) ABSTRACT

A single-use cartridge, wherein the cartridge defines a plurality of blind bores, each blind bore defining one opening at least one of the plurality of blind bores containing two mutually immiscible liquids, both of which are immiscible with water, one of the two mutually immiscible liquids having a specific gravity greater than water and the other of the two mutually immiscible liquids having a specific gravity less than water.

### CROSS-REFERENCE TO RELATED APPLICATIONS

This application claims the benefit of U.S. provisional application Ser. No. 61/908,479 filed 25 Nov. 2013 and U.S. provisional application Ser. No. 61/908,489 filed 25 Nov. 2013, which are hereby incorporated herein by reference in its entirety.

### BACKGROUND

Current biochemistry processing methods, typically carried out in the macroscopic wells of multi-well plates, has a number of drawbacks that are addressed by the use of composite liquid cells, or CLCs. CLCs are described in U.S. provisional applications Ser. Nos. 61/344,434, filed Jul. 22, 2010, 61/470,515, filed Apr. 1, 2011, and 61/470,520, filed Apr. 1, 2011, and U.S. application Ser. No. 13/147,679, filed Aug. 3, 2011, each of which is hereby incorporated herein by reference in its entirety. CLCs allow for biochemical protocols, such as nucleic acid amplification, to be carried out using far smaller quantities of reagents than would be required for processing in a standard multi-well plate. Use of CLCs also allows for ease of processing, since CLCs can be moved, combined, and divided with relative ease.

### SUMMARY

Devices, systems, and methods for CLCs are disclosed.

### BRIEF DESCRIPTION OF THE DRAWINGS

FIG. 1 schematically shows a cartridge defining a plurality of blind bores.

FIG. 2 schematically shows the cartridge of FIG. 1 in cross-section, empty.

FIG. 3 schematically shows the cartridge of FIG. 1 in cross-section, with oils and reagents sealed in each blind bore, and includes a close-up of one bore

FIG. 4 schematically shows a plate made of a plurality of cartridges as shown in FIG. 1, shown without seal for clarity.

FIG. 5 schematically shows the plate of FIG. 4 with seals covering the blind bores.

FIG. 6 depicts an embodiment of a system of the invention. Oil A and Oil B are placed into a conduit, which is open at the top and the bottom. Either opening is capable of being closed. The conduit may be in the shape of a conical frustum. Oil B has a higher density than Oil A, and the oils are immiscible. A sample can be added to the conduit. The sample has a density intermediate between the density of Oil A and the density of Oil B. Furthermore, the sample is substantially immiscible with both Oil A and Oil B. The sample, Oil A, and Oil B may be added to the conduit in any order.

FIG. 7 depicts a method of dispensing the sample from the system. Oil B is dispensed from the system through the bottom opening of the conduit. A volume of the sample is dispensed from the system through the bottom opening of the conduit. This may be accomplished by applying positive pressure to the top opening of the conduit. Oil B is returned to the conduit. This may be accomplished by applying negative pressure to the top of the conduit while the bottom of the conduit is contacting Oil B.

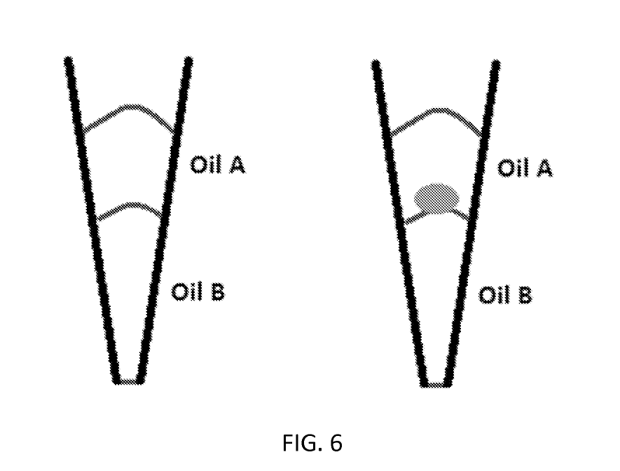

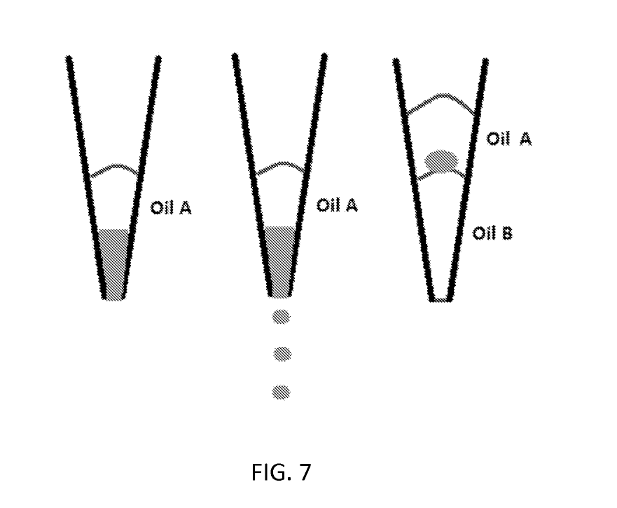

Note that FIGS. 6 and 7 show every meniscus being convex, i.e., bulging upwards toward a central maximum, as would be the case for a typical oil in a polypropylene vessel. If the vessel and/or liquid were differently constituted and had different relative wetting properties, the meniscus could be convex, i.e., bulging downward toward a central minimum, as would be the case for many oils in a vessel having a polytetrafluoroethylene inner surface.

### DETAILED DESCRIPTION

CLCs are generally formed by a combination of three substantially mutually immiscible liquids having three different densities. At the bottom is a carrier fluid, the densest of the three, and at the top is an encapsulating fluid, the least dense. Between the carrier and the encapsulating fluids is the liquid being contained in the CLC, typically aqueous, which can be, for example, a biological sample or a reagent, buffer, or other prescribed element of a biochemical protocol. This general arrangement of three mutually immiscible liquids can be advantageously applied in situations other than manipulation for use in a biochemical protocol, for example, storage in a blind bore in a transportable cartridge, or storage in and dispensing from a device having a through bore, e.g., an open-ended conduit.

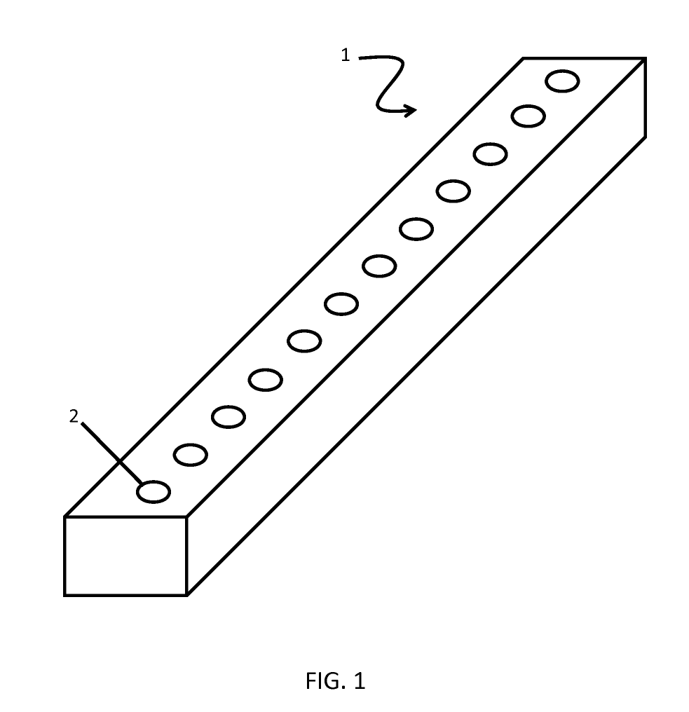

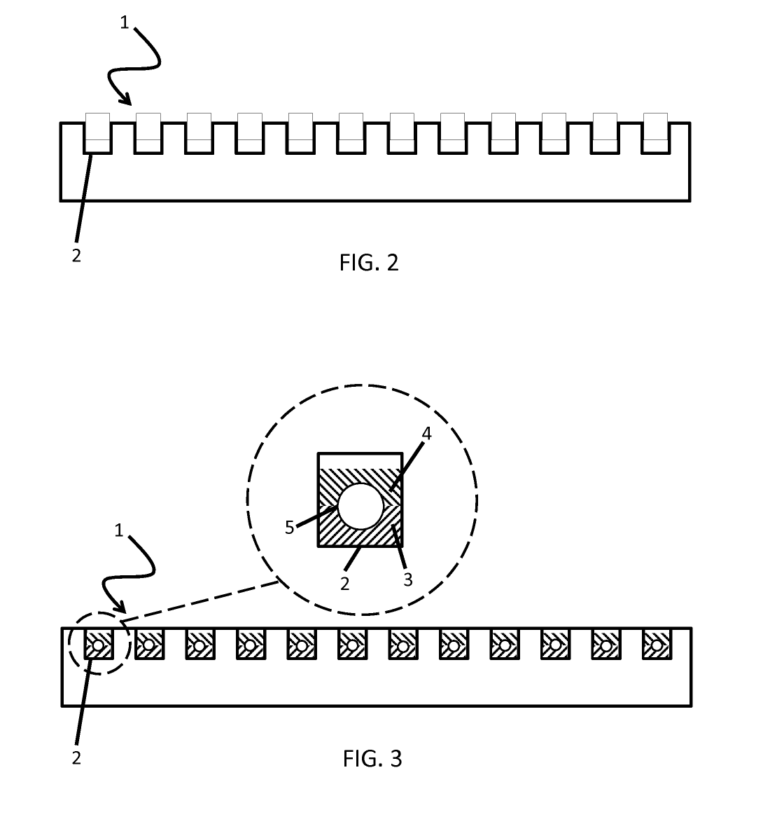

FIG. 1 schematically shows a cartridge 1 defining a plurality of blind bores. The blind bores are co-linear, arranged in a line along the length of the cartridge 1. A first blind bore 2 is positioned at one end of the cartridge 1. FIG. 2 shows the same cartridge 1 in cross-section. As shown, the bores are empty. FIG. 3 shows the same cross-section of the same cartridge 1 as FIG. 2, but in this case the bores are shown filled with fluid. In some embodiments, one or more bores will contain three fluids having three different densities or specific gravities, all three fluids being mutually immiscible. At the bottom of the bore 2 is a high density fluid 3 shown in cross-hatching, for example Fluorinert FC-40 (fluorocarbonated oil) having a density of approximately 1.9 g/cc. Above the high density fluid 3 is a mutually immiscible low density fluid 4 shown in opposite cross-hatching, for example phenylmethylpolysiloxane (silicone oil) having a density of approximately 0.92 g/cc. Between these two oils is an aqueous element 5 having a density of about 1.0 g/cc. Both oils and the aqueous element are all substantially mutually immiscible, giving the contents of each bore many of the functionalities of a CLC.

In some embodiments one or more bores can be left entirely empty, while others include one or more fluids, e.g., oils. In some embodiments, some bores or all the bores may include only the two oils, in anticipation of the addition of an aqueous element. The remaining bores may contain both oils and aqueous elements containing reagents necessary to carry out a biochemical protocol, for example an assay for the presence of a particular bioagent, or the reagents necessary to amplify nucleic acids by PCR. In any case, a sealed cartridge (as discussed below) may be shipped with a CLC-sized quantity of the necessary reagents included in one or more bores. Because the amount of reagent required is so small, the cost of such a cartridge can be kept low. The presence of the two mutually immiscible oils surrounding an aqueous solution also provides stability for the aqueous solution, since the aqueous elements are safely encapsulated in the equivalent of an immobilized CLC.

In some embodiments some or all aqueous components or reagents are dried, e.g., lyophilized or freeze dried, to enhance stability and transport.

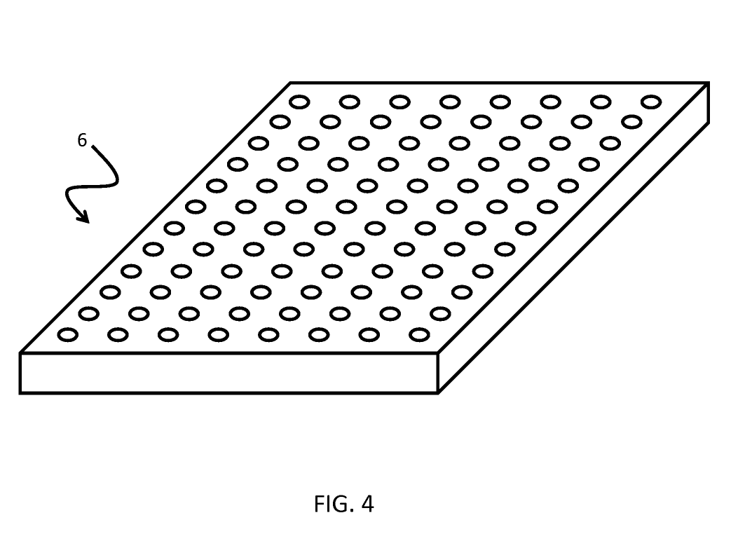

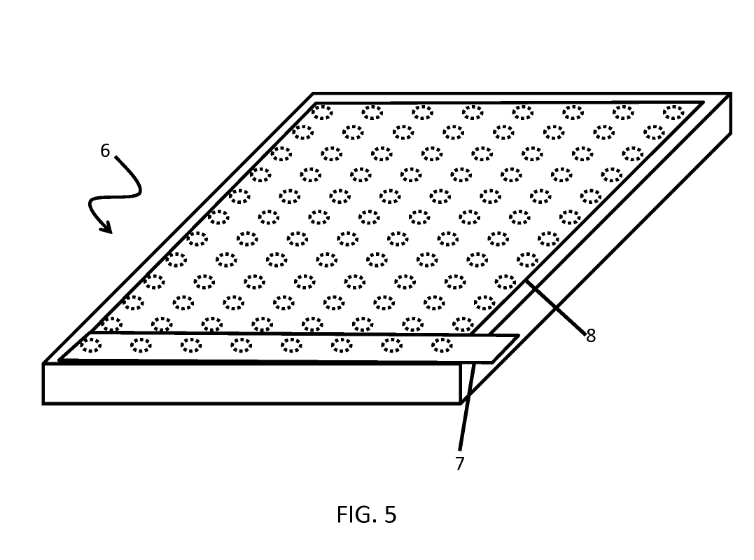

FIGS. 4 and 5 show a series of cartridges, like the ones shown in FIGS. 1-3, assembled together to make a single plate 6. Each row of bores corresponds to a single cartridge. The plate can be made of previously separate cartridges that have been affixed to one another, or the plate can be an integrally formed single piece (in such an embodiment, description herein of the features of a cartridge also apply to a row or section of a unitary or integrally-formed plate). FIG. 5 shows the plate sealed. The first bore in each cartridge is sealed with a tear-away film 7. In this embodiment, the film 7 is intended to be opened by hand by a user, and samples are to be inserted into the first row of bores, i.e., the first bore in each cartridge. The rest of the bores are covered with a single seal 8. In this case the seal 8 is a pierceable film. When a cartridge 1 or plate 6 is used, a liquid handling system may access the liquids inside the bores by piercing the seal 8. The positions of the bores beneath the film 7 and the seal 8 are shown in dotted lines. Although the first bore is shown at one end of the plate/cartridge, it could be positioned anywhere. Any bore could be designated as the bore intended to receive a sample, or be considered the “first” bore. The seal may be any suitable material, e.g., metallic foil or polymeric film or combinations thereof.

A single use cartridge can include a seal and define a plurality of co-linear blind bores, each blind bore defining one opening, at least one of the plurality of blind bores containing two mutually immiscible liquids, both of which are immiscible with water, one of the two mutually immiscible liquids having a specific gravity greater than water and the other of the two mutually immiscible liquids having a specific gravity less than water. The seal can cover the opening defined by the at least one blind bore, thereby making the interior of the blind bore substantially leak proof to the two mutually immiscible liquids.

In some embodiments all the blind bores contain the same two mutually immiscible liquids. In some embodiments at least one blind bore contains only the two mutually immiscible liquids.

In some embodiments the cartridge may have a first blind bore that is (a) positioned at one end of the line of the plurality of blind bores, and (b) is sealed with a portion of the seal adapted to be torn off by a human user by hand thereby exposing the opening of the first blind bore. In some embodiments the first blind bore contains only the two mutually immiscible liquids. In some embodiments at least one of the plurality of blind bores other than the first blind bore contains a reagent that is in an aqueous solution, wherein the reagent is an element of a predetermined biochemical protocol. In some embodiments all the reagents necessary to carry out the predetermined biochemical protocol are separately contained in the plurality of blind bores other than the first blind bore. In some embodiments the predetermined biological protocol is nucleic acid amplification by PCR. In some embodiments at least a portion of the seal is a pierceable film closing the opening defined by at least one blind bore. Optionally, one or more of such reagents are dried, and some bores may be empty to allow for mixing or waste disposal.

In some embodiments the plate can include a plurality of cartridges, the plate being formed by connection or other integration of the plurality of cartridges, the cartridges being arranged in the plate so that each plurality of co-linear blind bores is parallel to all the others. Alternatively, such a plate can be formed with the blind bores in any other pattern, e.g., by use of cartridges having other configurations. As noted, the plate may also be a single, unitary piece.

An embodiment of the system of the invention can include such a cartridge or plate; a cartridge or plate receiver sized and shaped to mate with the cartridge or plate; a liquid handling system capable of piercing the pierceable film closing any of the plurality of blind bores by inserting a liquid conduit into any of the blind bores when the cartridge has been mated with the cartridge receiver; and a controller operably connected to the liquid handling system. The controller can be programmed to, for example, cause the liquid handling system to: form a composite liquid cell in one of the blind bores; and operate upon the composite liquid cell and the reagents so as to carry out the predetermined biochemical protocol.

In some embodiments the system includes a thermal cycler. In some embodiments the system includes a cooling unit. In some embodiments the system includes optical fluorescence excitation and detection module. In some embodiments the system includes a capillary tube. In some embodiments the system includes a magnetic separation module.

A method of carrying out a biochemical protocol can include providing a system for using a cartridge or plate as described above; inserting the cartridge or plate into the receiver; depositing a biological sample in a first blind bore (either before or after inserting the cartridge or plate into the receiver); activating the controller so that the controller causes the liquid handling system to: form a composite liquid cell that contains an aqueous solution of the biological sample, and operate upon the composite liquid cell and the reagents so as to carry out the predetermined biological protocol on the biological sample.

In addition to systems in which biological samples or reagents are stored in a blind bore, this disclosure also provides in some embodiments systems and methods for the storage and dispensing of a sample from a conduit such as a through bore. The conduit can have flow in either direction and is controlled by a controller. In certain embodiments, a sample, such as a reagent or a biomolecule, is suspended between two fluids, such as oils. In certain embodiments, the sample is used as required and then re-suspended once the required volume of sample is dispensed. In certain embodiments, if a plurality of units of sample is required, the sample may be resuspended after each unit is dispensed, or after a number of units are dispensed, or after all required units of sample are dispensed.

### Exemplary Embodiments

In certain embodiments, the invention relates to a system including a conduit, a pump, a first fluid (e.g., fluid A), a second fluid (e.g., fluid B), and a sample. The conduit can define a top opening and a bottom opening, and can include an internal surface disposed between the top opening and the bottom opening. The first fluid, the sample, and the second fluid can be disposed within the conduit between the top opening and the bottom opening. The first fluid, the second fluid and the sample can be substantially mutually immiscible. The first fluid can be less dense than the sample and the sample can be less dense than the second fluid. The sample can be disposed between the first fluid and the second fluid such that the entire surface area of the sample is in fluid contact with only the first fluid, the second fluid, or the internal surface of the conduit. In some embodiments a pump can configured to apply positive pressure, negative pressure, or no external pressure to a location in the conduit.

In certain embodiments, the invention relates to any one of the systems described herein, wherein the system consists essentially of a conduit such as a through bore, a first fluid (e.g., fluid A), a second fluid (e.g., fluid B), and a sample, and optionally a pump.

In certain embodiments, the invention relates to any one of the systems described herein, wherein the system includes a means for temporarily sealing or temporarily closing the bottom opening of the conduit.

In certain embodiments, the invention relates to any one of the systems described herein, wherein the system includes a means for temporarily sealing or temporarily closing the top opening of the conduit.

In certain embodiments, the invention relates to any one of the systems described herein, wherein the sample is in an aqueous solution.

In certain embodiments, the invention relates to any one of the systems described herein, wherein the sample includes a reagent. Examples of reagents include those useful in sequence bead preparation, pyrosequencing, nucleic acid ligation, and polymerase chain reaction. In certain embodiments, the reagent is on a bead.

In certain embodiments, the invention relates to any one of the systems described herein, wherein the sample includes a biomolecule. Examples of biomolecules include (and are not limited to) cells, nucleic acids, proteins, enzymes, blood, saliva, and organic material. In certain embodiments, the biomolecule is on a bead.

In certain embodiments, the invention relates to any one of the systems described herein, wherein the sample includes more than one reagent or more than one biomolecule, or a reagent and a biomolecule.

In certain embodiments, the invention relates to any one of the systems described herein, wherein the first fluid is immiscible with the second fluid.

In certain embodiments, the invention relates to any one of the systems described herein, wherein the first fluid is immiscible with the sample.

In certain embodiments, the invention relates to any one of the systems described herein, wherein the second fluid is immiscible with the sample.

In certain embodiments, the invention relates to any one of the systems described herein, wherein the first fluid or the second fluid is an oil.

In certain embodiments, the invention relates to any one of the systems described herein, wherein the first fluid is an oil.

In certain embodiments, the invention relates to any one of the systems described herein, wherein the second fluid is an oil.

In certain embodiments, the invention relates to any one of the systems described herein, wherein the oils used for generating immiscible phases, for example the first fluid or the second fluid, can include and are not limited to silicone oil, perfluorocarbon oil, and perfluoropolyether oil.

In certain embodiments, the invention relates to any one of the systems described herein, wherein typical values of densities for the fluids involved range within the values about 1,300 to about 2,000 kg/m³ for the second fluid, about 700 to about 990 kg/m³ for the first fluid, and about 900 to about 1200 kg/m³ for the sample. An example of one such set of operating fluids and densities is outlined herein but is not limited to these; the second fluid is Fluorinert FC-40 (fluorocarbonated oil) density of approximately 1.900 kg/m³; the first fluid is phenylmethylpolysiloxane (silicone oil) density of approximately 920 kg/m³; and the sample is an aqueous based solution of PCR reagents with a density of approximately 1000 kg/m³.

In certain embodiments, the invention relates to any one of the systems described herein, wherein the second fluid is a perfluorinated amine oil.

In certain embodiments, the invention relates to any one of the systems described herein, wherein the first fluid is a solution of a phenylmethylpolysiloxane-based oil and a polysorbate additive. The additives have a hydrophilic-lipophilic balance number in the range of 2 to 8. The combined total hydrophilic-lipophilic balance number of the additives is in the range of 2 to 8. Examples of polysorbate additives are sorbitane monooleate, sorbitane tristearate, and polysorbate 20 but are not limited to these. These additives within the first fluid range between 0.001% and 10%.

In certain embodiments, the invention relates to any one of the systems described herein, wherein the sample is a solid particle suspension in aqueous media, the first fluid is a phenylmethylpolysiloxane-based oil, and the second fluid is a fluorocarbon-based oil.

In certain embodiments, the invention relates to any one of the systems described herein, wherein the sample is an aqueous media-in-phenylmethylpolysiloxane-based oil, the first fluid is a phenylmethylpolysiloxane-based oil, and the second fluid is a fluorocarbon-based oil.

In certain embodiments, the invention relates to any one of the systems described herein, wherein the conduit is oriented substantially vertically.

In certain embodiments, the invention relates to any one of the systems described herein, wherein the conduit is oriented vertically.

In certain embodiments, the invention relates to any one of the systems described herein, wherein the conduit is a capillary tube.

In certain embodiments, the invention relates to any one of the systems described herein, wherein the conduit is in the shape of a conical frustum.

In certain embodiments, the invention relates to any one of the systems described herein, wherein the conduit is in the shape of a conical frustum; and the top opening of the conduit has an internal diameter that is larger than the internal diameter of the bottom opening of the conduit.

In certain embodiments, the invention relates to any one of the systems described herein, wherein the conduit is a pipette tip.

In certain embodiments, the invention relates to any one of the systems described herein, wherein the conduit is made from a polymer, ceramic, or metal.

In certain embodiments, the invention relates to any one of the systems described herein, wherein the conduit includes a hydrophobic surface.

In certain embodiments, the invention relates to any one of the systems described herein, wherein the conduit is a polymer capillary tube, such as a polytetrafluoroethylene (PTFE) capillary tube.

In certain embodiments, the invention relates to any one of the systems described herein, wherein the conduit is disposable.

In certain embodiments, the invention relates to any one of the systems described herein, wherein the conduit is reusable.

In certain embodiments, the invention relates to any one of the systems described herein, wherein the conduit has an internal diameter is typically within a range of from about 10 microns to about 10 millimetres in diameter.

In certain embodiments, the invention relates to any one of the systems described herein, wherein the internal diameter of the conduit is variable from the bottom of the conduit to the top of the conduit, along the length of the conduit.

In certain embodiments, the invention relates to any one of the systems described herein, wherein the conduit has a wall thickness of at least about 10 microns or more.

In certain embodiments, the invention relates to any one of the systems described herein, wherein the internal shape of the conduit can be (and is not necessarily limited to) a profile which is round, conical, square, oval, rectangular, have a wavy surface, have at least one flat surface, or have surface enhancement features.

In certain embodiments, the invention relates to any one of the systems described herein, wherein the external shape of the conduit can be (and is not necessarily limited to) a profile which is round, conical, square, oval, rectangular, have a wavy surface, have at least one flat surface, or have surface enhancement features.

In certain embodiments, the invention relates to any one of the systems described herein, wherein the pump is a vacuum pump.

In certain embodiments, the invention relates to any one of the systems described herein, wherein the pump is a pipette bulb.

In certain embodiments, the invention relates to any one of the systems described herein, wherein the pump is a means for applying positive pressure.

In certain embodiments, the invention relates to any one of the systems described herein, wherein the pump is a means for applying negative pressure.

In certain embodiments, the invention relates to any one of the systems described herein, wherein the pump is operably connected to the top of the conduit.

In certain embodiments, the invention relates to any one of the systems described herein, wherein the pump is operably connected to the bottom of the conduit.

In certain embodiments, the invention relates to any one of the systems described herein, wherein the system also includes a controller.

In certain embodiments, the invention relates to any one of the systems described herein, wherein the controller is operably connected to the pump.

In certain embodiments, the invention relates to any one of the systems described herein, wherein a plurality of conduits are assembled together in a cassette.

In certain embodiments, the invention relates to any one of the systems described herein, wherein a plurality of conduits are assembled together on a plate.

In certain embodiments, the invention relates to any one of the systems described herein, wherein the system is at about 0° C., about 3° C., about 5° C., about 10° C., about 15° C., about 20° C., about 25° C., about 30° C., about 35° C., about 37° C., about 40° C., or about 45° C.

In certain embodiments, the invention relates to any one of the systems described herein, wherein the system is a micropipette.

In certain embodiments, the invention relates to a method for storing a sample in a system, the method including:

providing a conduit, a pump, a first fluid, a second fluid, and a sample, wherein

the conduit has a top opening, a bottom opening, and an internal surface disposed between the top opening and the bottom opening;

the first fluid is substantially immiscible with the second fluid;

the first fluid is substantially immiscible with the sample;

the second fluid is substantially immiscible with the sample;

the first fluid is less dense than the sample;

the sample is less dense than the second fluid; and

the pump is configured to apply positive pressure, negative pressure, or no external pressure to a location in the conduit; and

loading into the conduit, in any order, the first fluid, the second fluid, and the sample, such that the first fluid, the sample, and the second fluid are disposed within the conduit between the top opening and the bottom opening, and the sample is disposed between the first fluid and the second fluid such that the entire surface area of the sample is in fluid contact with only the first fluid, the second fluid, or the internal surface of the conduit.

In certain embodiments, the invention relates to any one of the methods described herein, wherein the system consists essentially of a conduit, a pump, a first fluid (e.g., fluid A), a second fluid (e.g., fluid B), and a sample.

In certain embodiments, the invention relates to any one of the methods described herein, wherein the system includes a means for temporarily sealing or temporarily closing the bottom opening of the conduit.

In certain embodiments, the invention relates to any one of the methods described herein, further including the step of:

temporarily sealing or temporarily closing the bottom opening of the conduit before loading the first fluid, the second fluid, or the sample into the conduit.

In certain embodiments, the invention relates to any one of the methods described herein, wherein the system includes a means for temporarily sealing or temporarily closing the top opening of the conduit.

In certain embodiments, the invention relates to any one of the methods described herein, also including the step of:

temporarily sealing or temporarily closing the top opening of the conduit before loading the first fluid, the second fluid, or the sample into the conduit.

In certain embodiments, the invention relates to any one of the methods described herein, wherein the first fluid is loaded into the conduit before the sample or the second fluid.

In certain embodiments, the invention relates to any one of the methods described herein, wherein the sample is loaded into the conduit before the first fluid or the second fluid.

In certain embodiments, the invention relates to any one of the methods described herein, wherein the second fluid is loaded into the conduit before the first fluid or the sample.

In certain embodiments, the invention relates to any one of the methods described herein, wherein the first fluid is loaded into the conduit via the top opening of the conduit.

In certain embodiments, the invention relates to any one of the methods described herein, wherein the first fluid is loaded into the conduit via the bottom opening of the conduit.

In certain embodiments, the invention relates to any one of the methods described herein, wherein the second fluid is loaded into the conduit via the top opening of the conduit.

In certain embodiments, the invention relates to any one of the methods described herein, wherein the second fluid is loaded into the conduit via the bottom opening of the conduit.

In certain embodiments, the invention relates to any one of the methods described herein, wherein the sample is loaded into the conduit via the top opening of the conduit.

In certain embodiments, the invention relates to any one of the methods described herein, wherein the sample is loaded into the conduit via the bottom opening of the conduit.

In certain embodiments, the invention relates to any one of the methods described herein, wherein the step of loading the first fluid, the second fluid, or the sample into the conduit via the bottom opening of the conduit includes applying negative pressure to the top opening of the conduit.

In certain embodiments, the invention relates to any one of the methods described herein, wherein the sample is in an aqueous solution.

In certain embodiments, the invention relates to any one of the methods described herein, wherein the sample includes a reagent.

In certain embodiments, the invention relates to any one of the methods described herein, wherein the sample includes a biomolecule. Examples of biomolecules include (and are not limited to) cells, nucleic acids, proteins, enzymes, blood, saliva, and organic material.

In certain embodiments, the invention relates to any one of the methods described herein, wherein the first fluid is immiscible with the second fluid.

In certain embodiments, the invention relates to any one of the methods described herein, wherein the first fluid is immiscible with the sample.

In certain embodiments, the invention relates to any one of the methods described herein, wherein the second fluid is immiscible with the sample.

In certain embodiments, the invention relates to any one of the methods described herein, wherein the first fluid or the second fluid is an oil.

In certain embodiments, the invention relates to any one of the methods described herein, wherein the first fluid is an oil.

In certain embodiments, the invention relates to any one of the methods described herein, wherein the second fluid is an oil.

In certain embodiments, the invention relates to any one of the methods described herein, wherein the oils used for generating immiscible phases, for example the first fluid or the second fluid, can include and are not limited to silicone oil, perfluorocarbon oil, and perfluoropolyether oil.

In certain embodiments, the invention relates to any one of the methods described herein, wherein typical values of densities for the fluids involved range within the values about 1.300 to about 2,000 kg/m³ for the second fluid, about 700 to about 990 kg/m³ for the first fluid, and about 900 to about 1200 kg/m³ for the sample. An example of one such set of operating fluids and densities is outlined herein but is not limited to these; the second fluid is Fluorinert FC-40 (fluorocarbonated oil) density of approximately 1,900 kg/m³; the first fluid is phenylmethylpolysiloxane (silicone oil) density of approximately 920 kg/m³; and the sample is an aqueous based solution of PCR reagents with a density of approximately 1000 kg/m³.

In certain embodiments, the invention relates to any one of the methods described herein, wherein the second fluid is a perfluorinated amine oil.

In certain embodiments, the invention relates to any one of the methods described herein, wherein the first fluid is a solution of a phenylmethylpolysiloxane-based oil and a polysorbate additive. The additives have a hydrophilic-lipophilic balance number in the range of 2 to 8. The combined total hydrophilic-lipophilic balance number of the additives is in the range of 2 to 8. Examples of polysorbate additives are sorbitane monooleate, sorbitane tristearate, and polysorbate 20 but are not limited to these. These additives within the first fluid range between 0.001% and 10%.

In certain embodiments, the invention relates to any one of the methods described herein, wherein the sample is a solid particle suspension in aqueous media, the first fluid is a phenylmethylpolysiloxane-based oil, and the second fluid is a fluorocarbon-based oil.

In certain embodiments, the invention relates to any one of the methods described herein, wherein the sample is an aqueous media-in-phenylmethylpolysiloxane-based oil, the first fluid is a phenylmethylpolysiloxane-based oil, and the second fluid is a fluorocarbon-based oil.

In certain embodiments, the invention relates to any one of the methods described herein, wherein the conduit is oriented substantially vertically.

In certain embodiments, the invention relates to any one of the methods described herein, wherein the conduit is oriented vertically.

In certain embodiments, the invention relates to any one of the methods described herein, wherein the conduit is a capillary tube.

In certain embodiments, the invention relates to any one of the methods described herein, wherein the conduit is in the shape of a conical frustum.

In certain embodiments, the invention relates to any one of the methods described herein, wherein the conduit is in the shape of a conical frustum; and the top opening of the conduit has an internal diameter that is larger than the internal diameter of the bottom opening of the conduit.

In certain embodiments, the invention relates to any one of the methods described herein, wherein the conduit is a pipette tip.

In certain embodiments, the invention relates to any one of the methods described herein, wherein the conduit is made from a polymer, ceramic, or metal.

In certain embodiments, the invention relates to any one of the methods described herein, wherein the conduit includes a hydrophobic surface.

In certain embodiments, the invention relates to any one of the methods described herein, wherein the conduit is a polymer capillary tube, such as a PTFE material capillary tube.

In certain embodiments, the invention relates to any one of the methods described herein, wherein the conduit is disposable.

In certain embodiments, the invention relates to any one of the methods described herein, wherein the conduit is reusable.

In certain embodiments, the invention relates to any one of the methods described herein, wherein the conduit has an internal diameter is typically within a range of from about 10 microns to about 10 millimetres in diameter.

In certain embodiments, the invention relates to any one of the methods described herein, wherein the internal diameter of the conduit is variable from the bottom of the conduit to the top of the conduit, along the length of the conduit.

In certain embodiments, the invention relates to any one of the methods described herein, wherein the conduit has a wall thickness of at least about 10 microns or more.

In certain embodiments, the invention relates to any one of the methods described herein, wherein the internal shape of the conduit can be (and is not necessarily limited to) a profile which is round, conical, square, oval, rectangular, have a wavy surface, have at least one flat surface, or have surface enhancement features.

In certain embodiments, the invention relates to any one of the methods described herein, wherein the external shape of the conduit can be (and is not necessarily limited to) a profile which is round, conical, square, oval, rectangular, have a wavy surface, have at least one flat surface, or have surface enhancement features.

In certain embodiments, the invention relates to any one of the methods described herein, wherein the pump is a vacuum pump.

In certain embodiments, the invention relates to any one of the methods described herein, wherein the pump is a pipette bulb.

In certain embodiments, the invention relates to any one of the methods described herein, wherein the pump is a means for applying positive pressure.

In certain embodiments, the invention relates to any one of the methods described herein, wherein the pump is a means for applying negative pressure.

In certain embodiments, the invention relates to any one of the methods described herein, wherein the pump is operably connected to the top of the conduit.

In certain embodiments, the invention relates to any one of the methods described herein, wherein the pump is operably connected to the bottom of the conduit.

In certain embodiments, the invention relates to any one of the methods described herein, wherein the system further includes a controller.

In certain embodiments, the invention relates to any one of the methods described herein, wherein the controller is operably connected to the pump.

In certain embodiments, the invention relates to any one of the methods described herein, wherein a plurality of conduits are assembled together in a cassette.

In certain embodiments, the invention relates to any one of the methods described herein, wherein a plurality of conduits are assembled together on a plate.

In certain embodiments, the invention relates to any one of the methods described herein, also including the step of storing the sample in the system for a period of time.

In certain embodiments, the invention relates to any one of the methods described herein, wherein the first period of time is on the order of minutes, days, months, or years.

In certain embodiments, the invention relates to any one of the methods described herein, also including the step of storing the sample in the system at a temperature of about 0° C., about 3° C., about 5° C., about 10° C., about 15° C., about 20° C., about 25° C., about 30° C., about 35° C., about 37° C., about 40° C., or about 45° C.

In certain embodiments, the invention relates to any one of the methods described herein, also including the step of storing the sample in the system for a period of time at a temperature of about 0° C., about 3° C., about 5° C., about 10° C., about 15° C., about 20° C. about 25° C., about 30° C., about 35° C., about 37° C., about 40° C. or about 45° C.

In certain embodiments, the invention relates to any one of the methods described herein, wherein the system is on an instrument.

In certain embodiments, the invention relates to any one of the methods described herein, wherein the system is a micropipette.

In certain embodiments, the invention relates to a method of dispensing a sample from any one of the aforementioned systems, including the step of

dispensing from the conduit via the bottom opening the second fluid; and

dispensing from the conduit via the bottom opening a volume of the sample.

In certain embodiments, the invention relates to any one of the methods described herein, also including the step of dispensing from the conduit the first fluid.

In certain embodiments, the invention relates to any one of the methods described herein, wherein the first fluid, the sample, or the second fluid is dispensed from the conduit by negative pressure applied by the pump to the conduit.

In certain embodiments, the invention relates to any one of the methods described herein, wherein the first fluid, the sample, or the second fluid is dispensed from the conduit by positive pressure applied by the pump to the conduit.

In certain embodiments, the invention relates to any one of the methods described herein, wherein the first fluid, the sample, or the second fluid is dispensed from the conduit by negative pressure applied by the pump to the bottom opening of the conduit.

In certain embodiments, the invention relates to any one of the methods described herein, wherein the first fluid, the sample, or the second fluid is dispensed from the conduit by positive pressure applied by the pump to the top opening of the conduit.

In certain embodiments, the invention relates to any one of the methods described herein, also including the step of reloading into the conduit the second fluid.

In certain embodiments, the invention relates to any one of the methods described herein, wherein the second fluid is reloaded into the conduit via the bottom opening.

In certain embodiments, the invention relates to any one of the methods described herein, wherein the second fluid is reloaded into the conduit by negative pressure applied by the pump to the top opening of the conduit.

In certain embodiments, the invention relates to any one of the methods described herein, wherein the second fluid is reloaded into the conduit by positive pressure applied by the pump to the bottom opening of the conduit.

In certain embodiments, the invention relates to any one of the methods described herein, wherein the volume of the sample dispensed from the conduit is from about 10 nL to about 20 μL.

In certain embodiments, the invention relates to any one of the methods described herein, wherein the volume of the sample dispensed from the conduit is about 10 nL, about 20 nL, about 30 nL, about 40 nL, about 50 nL, about 60 nL, about 70 nL, about 80 nL, about 90 nL, about 100 nL, about 200 nL, about 300 nL, about 400 nL, about 500 nL, about 600 nL, about 700 nL, about 800 nL, about 900 nL, 1 μL, about 2 μL, about 3 μL, about 4 μL, about 5 μL, about 6 μL, about 7 μL, about 8 μL, about 9 μL, about 10 μL, about 11 μL, about 12 μL, about 13 μL, about 14 μL, about 15 μL, about 16 μL, about 17 μL, about 18 μL, about 19 μL, or about 20 μL.

In certain embodiments, the invention relates to any one of the methods described herein, wherein the volume of the sample dispensed from the conduit is a predetermined volume.

In certain embodiments, the invention relates to any one of the methods described herein, wherein the system includes a dead volume.

In certain embodiments, the invention relates to any one of the methods described herein, wherein the dead volume is from about 10 nL to about 200 nL.

In certain embodiments, the invention relates to any one of the methods described herein, wherein the dead volume is about 10 nL, about 20 nL, about 30 nL, about 40 nL, about 50 nL, about 60 nL, about 70 nL, about 80 nL, about 90 nL, about 100 nL, about 110 nL, about 120 nL, about 130 nL, about 140 nL, about 150 nL, about 160 nL, about 170 nL, about 180 nL, about 190 nL, or about 200 nL.

In certain embodiments, the invention relates to any one of the methods described herein, wherein the steps are automated.

In certain embodiments, the invention relates to any one of the methods described herein, wherein the steps are automated and controlled by the controller.

In certain embodiments, the invention relates to any one of the methods described herein, also including the step of cleaning or decontaminating the conduit.

In certain embodiments, the invention relates to any one of the methods described herein, wherein the conduit is cleaned or decontaminated with a cleaning agent.

In certain embodiments, the invention relates to any one of the methods described herein, wherein the conduit is reusable; and the cleaning agent is steam.

In certain embodiments, the invention relates to any one of the methods described herein, wherein the conduit is reusable; and the cleaning agent is bleach.

In certain embodiments, the invention relates to any one of the methods described herein, wherein the conduit is reusable; and the cleaning agent includes an enzyme.

In certain embodiments, the invention relates to any one of the methods described herein, wherein the conduit is reusable; and the cleaning agent includes a DNA digestion enzyme.

In certain embodiments, the invention relates to any one of the methods described herein, wherein the cleaning agent includes bleach, a base, a detergent, a surface disinfectant, a mild detergent, a mild acid, or a disinfectant.

Applications

Composite Liquid Cell Processing

In one embodiment, the sample is dispensed into an immiscible fluid cell positioned on a free surface of a mutually immiscible fluid. The resulting composite fluid cell can be transported, and/or merged, and/or mixed, and/or have biochemical processing performed on it.

In one embodiment, the sample is dispensed into an immiscible fluid cell positioned on a free surface of a mutually immiscible fluid with a mechanical stabilization feature.

In one embodiment, the upon dispensing from the conduit onto a free surface, the second fluid, the sample, and the first fluid generate a composite liquid cell.

In one embodiment, the sample has paramagnetic beads and buffer.

In one embodiment, the sample contains a buffer.

Examples of composite liquid cell systems to which the present systems and methods can be adapted are disclosed, for example, in PCT/IE2011/000040, which is hereby incorporated herein by reference.

Sequencing

Many next generation sequencing (NGS) platforms require DNA libraries made up of DNA fragments within a specific range of base pair lengths. In addition, these DNA fragments need to be tagged with specific nucleotide sequences (adapters) to allow the sequences to be amplified using PCR and to allow the library fragments to anneal to the sequencer flow cell. Sequence specific indices can also be added to the DNA fragments to identify individual samples when multiplexing sample within a single flow cell. The tagmentation of DNA (DNA is fragmented and tagged with adapters) and the addition of common adapters and indices is achieved in two separate biological reactions. Following these reactions, the DNA library is cleaned to remove excess nucleotides, enzymes, primers, salts and other contaminants. Consequently, the workflow required to tagment DNA, purify tagmented DNA, add common adapters and indices and purify the final library product is complex and labour intensive. In one embodiment the aforementioned systems and methods can be used to automate genetic sequencing.

Genetic Sequencing Bead Coating

Genetic sequencing bead preparation is a process by which small beads are coated in an application-specific chemistry. In one embodiment the coating of beads in advance of genetic screening is achieved by the systems and methods of the invention.

These methods provide for a convenient way of manipulating and combining sub-microlitre volumes of fluid that is currently not possible to achieve using conventional techniques, thereby reducing the initial sample volumes and improving the bead coating efficiency by reducing the reaction volume. Further processing using PCR and thermal cycling and genetic sequencing is application-specific.

The use of this technology greatly simplifies the collection procedure for these relatively small target volumes. The system facilitates 100% volume retrieval as the biological sample in processing does not incur any pipetting loses. These features make automation of the biochemistry process easier to facilitate.

Size Selection of DNA Libraries for NGS Sequencing

Each of the next generation sequencers have an optimal read length (base pairs). During library construction, DNA is fragmented into DNA molecules with a wide base pair length range. Size selection is currently performed using paramagnetic beads on a microtitre plate and is labour intensive and suffers from inefficiencies from pipetting errors and user protocol variations. The systems and methods of the invention can be used for size selection of DNA libraries.

Nucleic Acid Purification

The systems and methods of the invention can be used for purification and/or isolation of samples before and/or after PCR.

Genotyping

The systems and methods of the invention can be used for the storage and dispensing of genotyping reagents or samples.

Compound Screening

The systems and methods of the invention can be used for the storage and dispensing of drug compound screening reagents or samples.

### EXAMPLES

The following examples illustrate particular embodiments, but should not be viewed as limiting the scope of the disclosed subject matter.

### Example 1

A number of chambers or wells, for example from 1 to 384 chambers, are filled with two immiscible oils such that one oil sits on top of the other (see FIG. 1). Each chamber defines an opening in the bottom. The chambers may be arrayed on a store plate. Reagent is added to the chambers and lies at the interface between the two oils, forming what in some cases will be a Composite Liquid Cell, or CLC (Composite Liquid Cells are described in detail in U.S. Pat. No. 8,465,707, which is hereby incorporated by reference in its entirety). In this way, the reagent can be stored at room temperature for long periods of time with no degradation to the reagent from freezing and/or thawing. As and when required the lower oil, oil B, is removed by positively pressurizing the chamber above oil A and forcing the lower oil out of the bottom of the chamber. The reagent then moves down to the orifice and is ready to be dispensed (see FIG. 2). Using the pressure dispensing method, droplets can be created and dispensed directly from the store plate in parallel from the plurality of chambers in the store plate. Droplets can be dispensed into another CLC, well plate, or to any chamber or fluid as required. Once the reagent has finished dispensing it can be returned to storage by drawing an aliquot of oil B and again suspending the reagent as a droplet between Oil B and Oil A. There are no issues with contamination as it is the same reagent used during the entire process and therefore can be repeated any number of times until the reagent runs out. The plate can be loaded at any time, discarded, or cleaned and reused.

1. A single-use cartridge, wherein:
the cartridge defines a plurality of blind bores, each blind bore defining one opening, at least one of the plurality of blind bores containing two mutually immiscible liquids, both of which are immiscible with water, one of the two mutually immiscible liquids having a specific gravity greater than water and the other of the two mutually immiscible liquids having a specific gravity less than water.

2. The cartridge of claim 1 further comprising a seal that covers the opening defined by the plurality of blind bores, thereby making the interiors of the blind bores substantially leak proof to the two mutually immiscible liquids.

3. The cartridge of claim 2 wherein a first blind bore is sealed with a portion of the seal adapted to be torn off by a human user by hand thereby exposing the opening of the first blind bore.

4. The cartridge of claim 2 wherein at least a portion of the seal is a pierceable film closing the opening defined by at least one blind bore.

5. The cartridge of claim 1 wherein the plurality of blind bores are co-linear.

6. The cartridge of claim 1 wherein all the blind bores contain at least the same two mutually immiscible liquids.

7. The cartridge of claim 1 wherein at least one blind bore contains only the two mutually immiscible liquids.

8. The cartridge of claim 1 wherein a first blind bore contains only the two mutually immiscible liquids.

9. The cartridge of claim 8 wherein at least one of the plurality of blind bores other than the first blind bore contains a reagent that is in an aqueous solution, wherein the reagent is an element of a predetermined biochemical protocol.

10. The cartridge of claim 9 wherein all reagents necessary to carry out the predetermined biochemical protocol are separately contained in the plurality of blind bores other than the first blind bore.

11. The cartridge of claim 10 wherein the predetermined biological protocol is nucleic acid amplification by PCR.

12. A multi-well plate consisting of a plurality of cartridges, each cartridge according to claim 1, the plate being integrally formed of the plurality of cartridges, the cartridges being arranged in the plate so that each plurality of co-linear blind bores is parallel to all the others.

13. A system comprising:
a cartridge according to claim 9;
a cartridge receiver sized and shaped to mate with the cartridge;
a liquid handling system capable of piercing the pierceable film closing any of the plurality of blind bores by inserting a liquid conduit into any of the blind bores when the cartridge has been mated with the cartridge receiver; and
a controller operably connected to the liquid handling system, the controller programmed to cause the liquid handling system to:
form a composite liquid cell in one of the blind bores; and
operate upon the composite liquid cell and the reagents so as to carry out the predetermined biochemical protocol.

14. The system of claim 13 further comprising a thermal cycler.

15. The system of claim 13 further comprising a cooling unit.

16. The system of claim 13 further comprising an optical fluorescence excitation and detection module.

17. The system of claim 13 wherein the liquid conduit comprises a capillary tube.

18. The system of claim 13 wherein the liquid conduit comprises a magnetic separation module.

19. A method of carrying out a biochemical protocol comprising:
providing a system according to claim 13;
depositing a biological sample in the first blind bore;
inserting the cartridge into the cartridge receiver;
activating the controller so that the controller causes the liquid handling system to:
form a composite liquid cell that contains an aqueous solution of the biological sample; and
operate upon the composite liquid cell and the reagents so as to carry out the predetermined biological protocol on the biological sample.

20. The method of claim 19 wherein the predetermined biological protocol is designed to carry out one of (a) library preparation, (b) nucleic acid sequencing, (c) PCR, (d) digital PCR, and (e) qPCR.

21-92. (canceled)
